(19) United States
(12) Patent Application Publication
(10) Pub. No.: US 2006/0265037 A1
(43) Pub. Date: Nov. 23, 2006

(54) Construction of cylindrical multicontact electrode lead for neural stimulation and method of making same

(76) Inventor: Janusz A. Kuzma, Parker, CO (US)

Correspondence Address: ADVANCED BIONICS CORPORATION, 25129 RYE CANYON ROAD, VALENCIA, CA, 91355, US

(21) Appl. No.: 10/289,719

(22) Filed: Nov. 6, 2002

Related U.S. Application Data
(60) Provisional application No. 60/338,166, filed on Nov. 13, 2001.

(51) Int. Cl. A61N 1/00

(52) U.S. Cl. 607/116; 607/117

(57) ABSTRACT

An implantable lead includes a tubular multi-helix wire-wound body having a plurality of helically wound wires embedded within a wall of the tube body. A ring contact, e.g., a platinum ring contact, is electrically and mechanically connected to at least one of the plurality of helically wound wires at at least one end of the multi-helix wire-wound body. In one embodiment, ring contacts are attached at both ends of the multi-helix wire-wound body. A lumen passes longitudinally through the center of the lead body.

The present application claims the benefit of U.S. Provisional patent application Ser. No. 60/338,166, filed Nov. 13, 2001.

### BACKGROUND OF THE INVENTION

The present invention relates to neural stimulation leads, and more particularly to a cylindrical multi-contact electrode lead and methods of making such a lead.

### SUMMARY OF THE INVENTION

The present invention is directed to the design and manufacturing technology of a low cost, multi-contact, implantable electrode lead for use in neurostimulation systems, such as a spinal cord stimulation system.

Most currently used leads are made with individually insulated wires that are helically wound and placed loosely within silicone or polyurethane tubing. A platinum contact is welded at the distal end of each wire, using a controlled spacing in between each contact. Voids between the contacts are then filled with a suitable polymer, such as silicone or polyurethane, using well-known injection molding techniques.

The present invention relates to a process of making an implantable multi-contact electrode lead that involves two main steps: (1) making a multi-helix lead having wires embedded in a polyurethane sheath or tube; and (2) assembling the multi-helix lead by connecting platinum contacts to the distal ends of the wires included in the multi-helix lead. Each of these two main steps may be further broken down into several sub-steps, or operations, as described in more detail below.

### BRIEF DESCRIPTION OF THE DRAWINGS

FIGS. 1A, 1B and 1C illustrate a first operation associated with making a multi-helix lead, i.e., coating each lead wire with polyurethane to the desired thickness.

FIGS. 2A and 2B depict a second operation associated with making the multi-helix lead, i.e., winding multiple wires on a mandrel that defines the desired diameter of the multi-helix lead;

FIGS. 3A, 3B and 3C show a third operation associated with making the multi-helix lead, i.e., fusing the polyurethane coating;

FIG. 4 shows a sectional view of a platinum ring that is used as an electrode contact, prior to attaching such contact to a wire within the multi-helix lead as part of the lead assembly;

FIG. 5 illustrates a first operation associated with the lead assembly, i.e., removing the insulation of selected wires at specified spacings at a distal end of the multi-helix lead in order to expose the respective wires to which the platinum contacts are to be attached;

FIG. 6 depicts a second operation associated with the lead assembly, i.e., the placement of a platinum contact over the exposed wire to which it is to be attached;

FIG. 7 shows a third operation associated with the lead assembly, i.e., using a special tool to make a “dimple” or indentation or “dink” in the platinum contact over the exposed wire; and

FIG. 8 shows the fourth operation associated with the lead assembly, i.e., using a laser to selectively melt the platinum contact in the fusion zone defined by the dimple so as to fuse the platinum with the underlying exposed wire.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

The invention is directed to an implantable neural stimulation lead having a multiplicity of electrode contacts at its distal end. A lumen passes through the center of the lead through which a stylet may be inserted, if needed, to aid during the implanting of the lead. A multiplicity of wires, at least one wire for each electrode contact, are helically wound and embedded within the lead body that surrounds the central lumen. At a proximal end of the lead, the wires may be connected to a connector that can be detachably secured to a mating connector of a pulse generator, or other electronic control unit. In some embodiments, the proximal end of the lead, including the wires, may be permanently connected (hard wired) to an electronic control unit, such as an implantable pulse generator.

The process of making the implantable neural stimulation lead of the present invention comprises two main steps.

The first step associated with the process of making the implantable neural stimulation lead is making a multi-helix lead body, i.e., a lead having multiple wires embedded in its body. The wires are helically wound so as to reside around the outer edge of the lead body, with a lumen passing centrally through the body. The helically wound wires are embedded in the lead body so as not to be exposed on the outer surface of the lead body, or on the inner surface lead body, i.e., so as not to be exposed within the lumen.

The second step associated with the process of making the implantable neural stimulation lead is to fashion, or to assemble, lead contacts at a distal end of the lead. Each lead contact is attached to a distal end of at least on the wires that pass through the lead body.

In addition to the two main steps outlined above, there is also a third step, at least for some embodiments of the invention, but not necessarily part of the invention (and therefore not described in any detail, but known in the lead-making art) of attaching a connector to a proximal end of the implantable neural stimulation lead. Such connector allows the proximal end of the lead to be mechanically and electrically connected to a suitable control unit, such as an implantable pulse generator (IPG), or to a lead extension that connects to an IPG. Alternatively, for some applications, a proximal end of the lead may be directly attached, without the use of a connector, to a suitable control unit, such as an IPG.

Note, for some other applications, the proximal end of the lead may be identical to the distal end of the lead. That is, the proximal end may have lead contacts attached to at least one of the wires passing through the lead body, as is the case at the distal end of the lead. In such instance, the contacts at the proximal end may be assembled just like the distal end, and the resulting lead contacts may be engaged with a suitable connector that forms part of, or is attachable to, an IPG.

The various operations associated with the first two steps of making implantable neural stimulation lead will next be described.

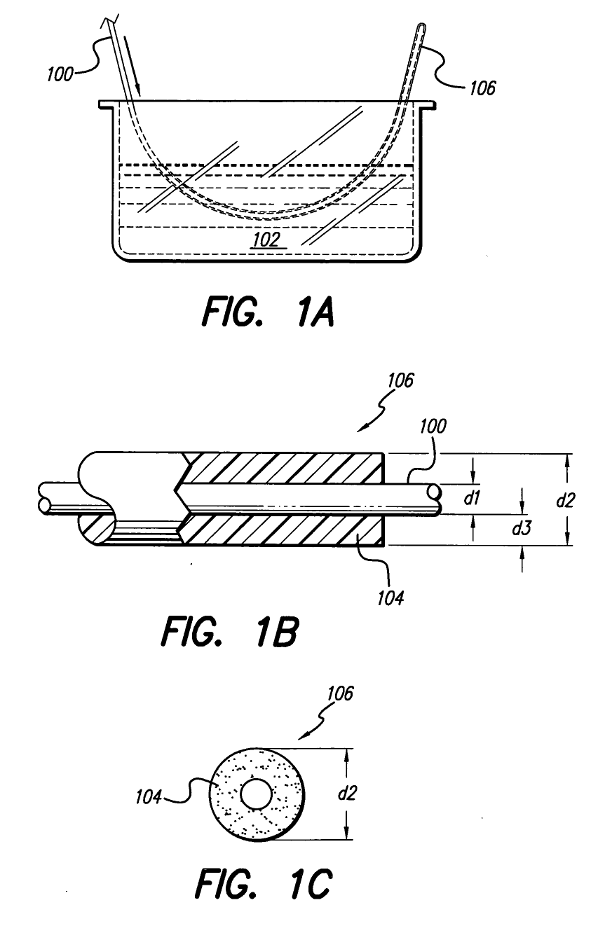

The first operation associated with the first step of making a multi-helix lead body comprises coating a lead wire with polyurethane to a desired thickness. Such process is illustrated in FIGS. 1A, 1B and 1C. In FIG. 1A, a wire 100 having a desired diameter d1 is drawn through liquid polyurethane 102, or equivalent coating material, in order to apply a coating 104 of polyurethane to the wire 100, thereby forming coated wire 106. When coated, the wire and coating have a combined diameter d2 (seem in FIG. 1C). The speed of drawing the wire 100 through the liquid polyurethane 102, and the temperature of the liquid polyurethane, determine in large part the thickness d3 of the coating. Assuming the coating is more of less uniform, the thickness d3 is thus equal to (d2−d1)/2. For a typical application, the wire diameter d1 is between about 0.1 and 0.2 mm; the coating thickness d3 is between about 0.05 and 0.15 mm; which means the overall diameter d2 of the coated wire 106 is between about 0.2 and 0.5 mm. The wire may be made from any suitable body compatible metal, such as platinum, titanium, stainless steel, or alloys thereof.

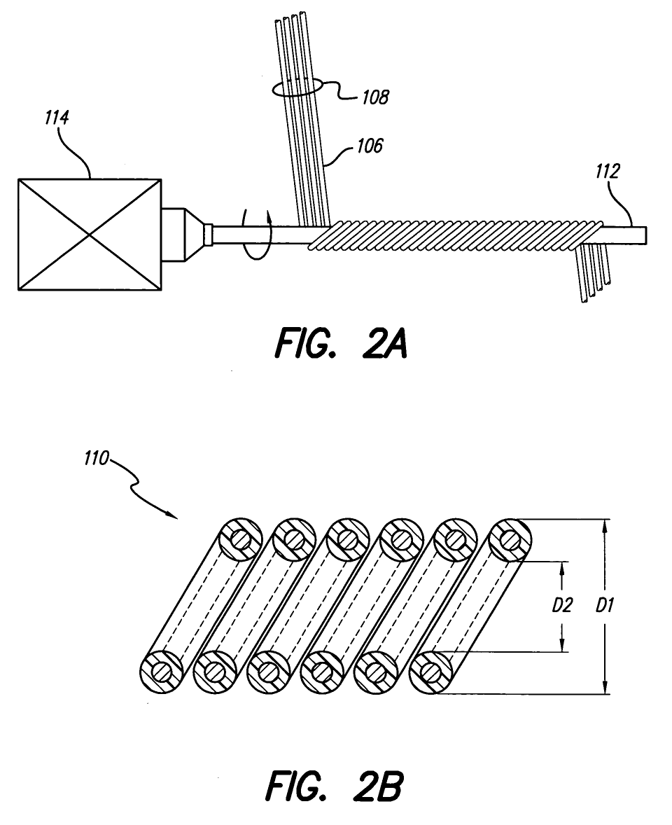

After the coated wire 106 is formed, then the second operation associated with the first step of making a multi-helix lead body comprises winding the coated wire to form a multi-helix body, as illustrated in FIGS. 2A and 2B. As illustrated in FIG. 2A, a multi-helix lead body 110 is formed by winding a plurality of coated wires 106 on a mandrel 112. Typically, such winding is accomplished by rotating the mandrel 112 with a suitable rotating member 114 while holding and positioning a group 108 of adjacent coated wires 106 so that such group of wires are wound on the mandrel 112. As the winding occurs, each wire 106 in the group of wires 108 is held tightly against an adjacent wire 106 of the group of wires 108. The result is that the multi-helix lead body 110, seen best in the sectional view of FIG. 2B, is formed having a prescribed diameter D1 and wire spacing P (also referred to as the wire pitch). The number of different helically-wound coated wires present in the multi-helix lead body is the number of wires in the group of wires 108. For the example illustrated in FIGS. 2A and 2B, four coated wires 106 are included within the multi-helix lead body 110. This number of wires is only exemplary, however, as any number of coated wires 106 could be included in the multi-helix body, e.g., from two to sixteen wires. After the winding of the wires on the mandrel is completed, the mandrel may be removed.

For the embodiment shown in FIGS. 2A and 2B, the outside diameter D1 of the multi-helix body 110 is between about 0.8 mm and 2.0 mm, and may (depending upon the application for which the lead is used) be between about 0.8 mm and 1.2 mm. The inner diameter D2 of the multi-helix body is between about 0.2 mm and 1.2 mm, and may (depending upon the application) be between about 0.2 mm and 0.4 mm). These dimensions are only exemplary.

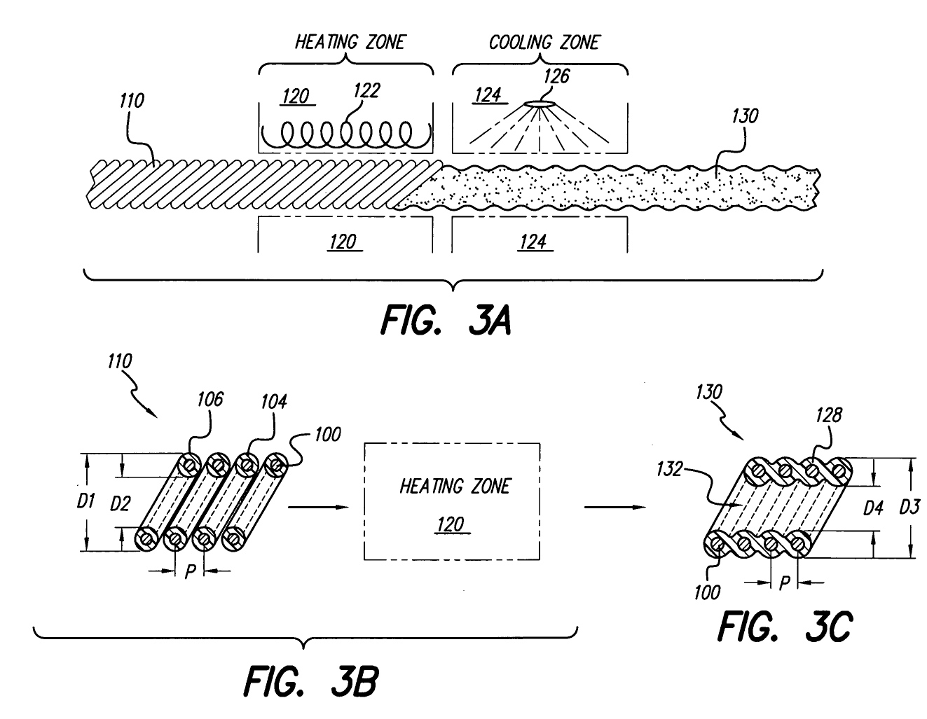

A third operation associated with the first step of making a multi-helix lead body comprises fusing the polyurethane coating of the coated wires 106. This third operation is schematically depicted in FIGS. 3A, 3B and 3C. As seen in FIG. 3A, the unfused multi-helix lead body 110 is driven with a controlled speed through a heating zone 120. The heating zone 120 elevates the temperature of the polyurethane coatings 104 on each wire to the melting point, causing such coatings to soften and fuse into a single jacket or sheath 128. The heating zone is followed by a rapid cooling zone 124.

The heat applied in the heating zone 120 may be supplied using any suitable heating source, such as an electric coil 122. The cooling applied in the cooling zone 124 may likewise be supplied using any suitable cooling source, such as a cooling liquid, e.g., water, applied through conventional cooling tubes and valves 126.

The result of the third operation shown in FIGS. 3A, 3B and 3C is that the unfused multi-helix body 110, seen, e.g., in FIG. 3B is converted to a more stable and completely sealed tubular polyurethane tubing 130, seen, e.g., in FIG. 3C. Advantageously, multiple wires 100, each of which is helically wound, are imbedded within the tubular polyurethane tubing 130. The desired wire spacing or pitch P is maintained in the multi-helix wire-wound tubing 130 that existed in the unfused multi-helix body 110. The outside diameter D3 of the fused tubing 130 is slightly reduced from the outside diameter D1 of the unfused body 110. Similarly, the inside diameter D4 of the fused tubing 130 is slightly increased from the inside diameter D2 of the unfused body 110.

Thus, it is seen that the fused multi-helix wire-wound tube 130 becomes very stable and has a well defined lumen 132 passing longitudinally through its center. Moreover, the fused tube 130 has an increased stiffness over the unfused multi-helix body 110, which increased stiffness helps with its handling and insertion. Nonetheless, in order to maintain a stiffness comparable to existing commercially available leads, the outside diameter D3 of the fused multi-helix wire-wound tube 130, for some applications, is preferably reduced from about 1.2 mm to about 0.8 mm.

A fourth operation associated with the first step of making a multi-helix lead body comprises inspecting the fused multi-helix wire-wound tube 130 and cutting it to a desired length.

Next, the second main step associated with the process of making the implantable neural stimulation lead is commenced. This second main step, as indicated previously, comprises fashioning, or assembling, and connecting lead contacts to a distal end of at least one of the wires that pass through the fused multi-helix wire-wound body 130. Like the first main step, this second step is also broken down into several operations.

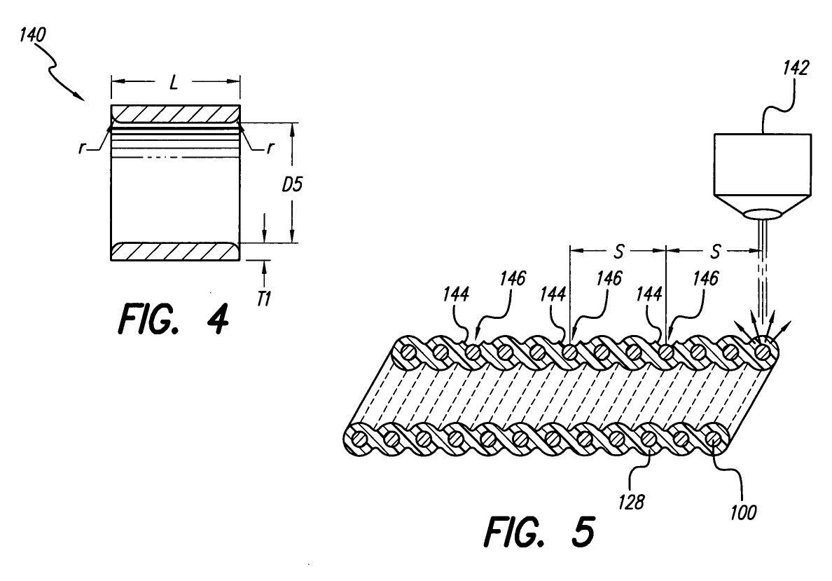

FIG. 4 illustrates a platinum ring contact 140 that may be attached to a distal end of the fused lead body. Such ring contact 140 may be made using standard manufacturing techniques known in the art. Depending upon the application, and the wire pitch spacing P that is used for that application, the ring contact 140 preferably has a length L of from between about 1.0 to 1.5 mm. The thickness T1 of the ring contact is about 0.05 mm. The inside diameter D5 of the ring contacts should be equal to or slightly smaller than the outside diameter D3 of the fused multi-helix wire wound tube 130. Thus, for one embodiment, where D3 is on the order of 0.8 mm, the inside diameter D5 of the ring contact 140 should be on the order of 0.75 mm. The internal edges of the ring contacts should be rounded, e.g., to have a radius “r”, to facilitate sliding of the contacts over the outer surface of the multi-helix wire-wound body 130.

Once the ring contacts 140 are formed, then a first operation associated with connecting the lead contacts to the multi-helix wire-wound body 130 comprises removing a small section of the fused 128 polyurethane coating. Such removal is done for the purpose of exposing one or more points of each helically-wound wire 106 within the multi-helix wire-wound body 130. Such removal may be accomplished various ways, but a preferred technique is to use a low power laser 142, e.g., a UV laser, to obliterate or ablate (i.e., remove) the top surface of the polyurethane coating so as to create a crater 144 in the polyurethane coating 128 that exposes one or more points 146 on each helically wound wire at a defined pitch or spacing S. This process is illustrated in FIG. 5.

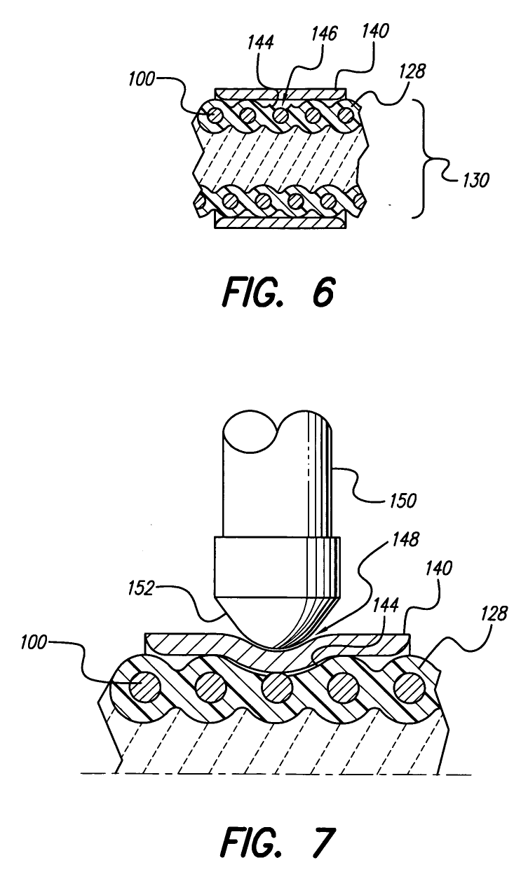

Next, a second operation associated with connecting the lead contacts to the multi-helix wire-wound body 130 comprises sliding the ring contacts 140 over the distal end of the lead body 130. As the contacts 140 are slid over the lead body, they are positioned at the desired spacing S above the points 146 where the wires are exposed, as depicted in FIG. 6.

A third operation associated with connecting the lead contacts to the multi-helix wire-wound body 130, depicted in FIG. 7, comprises creating at least one indentation 148, or dimple (also known as a “dink”), in the platinum ring contact 140 above the point 146 where the wires are exposed, i.e., above the location where the craters 144 were created on the surface of the coating 128. Such indentations are made using a suitable tool 150 having a tapered tip 152 configured to deform the contact 140 so that it fits within the crater 144 and makes contact with the exposed wire. Such process may be accomplished relatively easily using a holding fixture equipped with a pressing punch.

The operation shown in FIG. 7 is performed for each ring contact 140. For some applications, a ring contact may be desired on both ends of the multi-helix wire-wound body 130, i.e., on a distal end and on a proximal end. (The ring contacts on the proximal end, if used, can then interface with a suitable connector into which the proximal end of the lead may be detachably secured.)

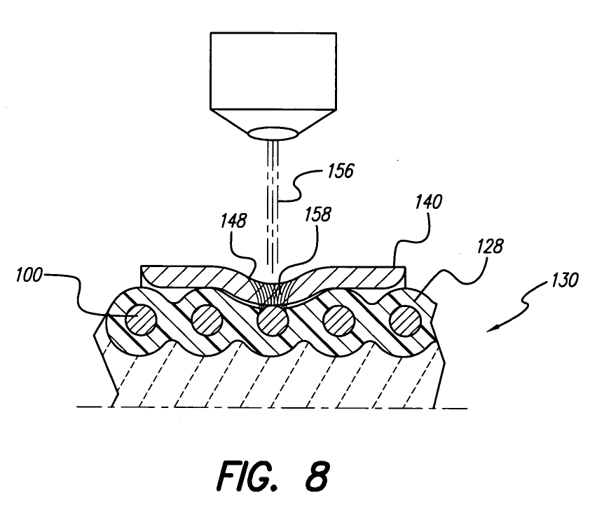

A fourth operation associated with connecting the lead contacts 140 to the multi-helix wire-wound body 130 comprises welding, or otherwise making a secure electrical and mechanical connection, between the lead contact 140 and the wire exposed at the point 146 beneath the ring contact. Such operation is preferably performed as illustrated in FIG. 8. As seen in FIG. 8, a YAG laser 154 is used to generated a laser beam 156. The laser beam 156 is directed to (or focused on) the indentation or dimple 148 formed in the platinum ring 140 above the point 146 where the wire in the multi-helix wire-wound body 130 is exposed. The heat associated with the laser beam 156 causes the platinum contact to melt, thereby creating a fusion zone 158 wherein the platinum contact 140 is fused with the underlying wire 100 in the multi-helix wire-wound body 130.

The fusing operation illustrated in FIG. 8 is performed on all contacts for at least one indentation, or dimple, associated with each contact.

Once the fusing operation has been performed, as illustrated in FIG. 8 for each contact, then a fifth operation involves trimming and sealing the end of the lead where the contacts are attached. If contacts 140 are attached to both the proximal and distal ends of the multi-helix wire-wound body 130, then trimming and perhaps sealing should be done at both ends of the lead body. (Sealing may only be needed at the proximal end of the lead, if a thin stylet is to be inserted in the lumen during placement of the lead.) If the lead contacts are attached to only the distal end of the lead body, then whatever connection needs to take place at the distal end can take place, e.g., directly connecting the proximal end of the lead to an IPG.

While the invention herein disclosed has been described by means of specific embodiments and applications thereof, numerous modifications and variations could be made thereto by those skilled in the art without departing from the scope of the invention set forth in the claims.

1. A method of making an implantable lead comprising:
coating a plurality of straight wires (100) with a polymer (102);
winding the plurality of wires on a mandrel to form a multi-helix body (110), wherein the multi-helix body has a prescribed wire pitch “P”, outer diameter D1, and inner diameter D2;
fusing the polymer coatings of the multi-helix body to form a tubular multi-helix wire-wound body (130) having a single wall, wherein the tubular multi-helix wire-wound body has the plurality of wires embedded within the single wall, wound in multiple helixes; and
attaching a plurality of ring contacts (140) to at least a distal end of the multi-helix wire-wound body, wherein each ring contact is mechanically and electrically connected to at least one of the plurality of wires embedded within the tubular multi-helix wire-wound body.

2. The method of claim 1 wherein fusing the polymer coatings comprises passing the multi-helix body through a heating zone (120) that raises the temperature of the polymer coatings surrounding the helically wound wires to a melting point of the polymer, thereby causing the coatings of each helically wound wire to fuse with the coatings of adjacent helically wound wires, and then immediately passing the multi-helix body through a cooling zone (124).

3. The method of claim 1 wherein attaching the plurality of ring contacts comprises removing the polymer coating from each wire so as to expose the wire at a connection point (146) near one end of the multi-helix wire-wound body; sliding the ring contact (140) so as to reside over the exposed wire; indenting the ring contact over the connection point to form an indentation, wherein the indentation forces an inner surface of the ring contact against the exposed wire at the connection point; and fusing the ring contact with the exposed wire at the connection point.

4. The method of claim 3 wherein fusing the ring contact with the exposed wire at the connection point comprises laser welding the ring contact to the exposed wire.

5. The method of claim 4 wherein laser welding the ring contact to the exposed wire comprises directing a laser beam to the indentation on the ring contact to create a fusion zone (158), wherein the ring contact at the fusion zone melts and fuses with the exposed wire.

6. The method of claim 5 further including forming the ring contacts from platinum.

7. The method of claim 6 wherein the polymer that coats the wires comprises polyurethane.

8.-19. (canceled)
